(19) United States
(12) Patent Application Publication
(10) Pub. No.: US 2019/0151212 A1
(43) Pub. Date: May 23, 2019

(54) PRODUCT FOR THE TEMPORARY RESHAPING OF KERATINOUS FIBRES

(71) Applicant: Henkel AG & Co. KGaA, Duesseldorf (DE)

(72) Inventors: Sandra Brandt, Pinneberg (DE); Nora Koopmann, Hamburg (DE); Marcus Noll, Quickborn (DE); Anna Puls, Winsen (Luhe) (DE)

(73) Assignee: Henkel AG & Co. KGaA, Duesseldorf (DE)

(21) Appl. No.: 16/193,237

(22) Filed: Nov. 16, 2018

(30) Foreign Application Priority Data
Nov. 23, 2017 (DE) 10 2017 220 993.3

(51) Int. Cl. A61K 8/35; A61K 8/37; A61K 8/362; A61K 8/36; A61K 8/34; A61Q 5/06

(52) U.S. Cl. CPC A61K 8/35; A61K 8/37; A61K 8/362; A61K 2800/524; A61K 8/342; A61Q 5/06; A61K 8/361

(57) ABSTRACT

The present disclosure relates to a cosmetic product for the temporary reshaping of keratinous fibres, particularly human hair, with good storage stability, and with increased microbiological stability and very good application properties, in particular including an enhancement of the natural lustre and the natural texture of the keratinous fibres.

### CROSS-REFERENCE TO RELATED APPLICATION

This application claims priority to German Patent Application No. 10 2017 220 993.3, filed Nov. 23, 2017, which is incorporated herein by reference in its entirety.

### TECHNICAL FIELD

The present disclosure relates to a cosmetic product for the temporary reshaping of keratinous fibres, and in particular for reshaping human hair.

### BACKGROUND

The temporary shaping of hairstyles for a prolonged period of up to several days typically requires the use of stabilising agents. This is why hair treatment products that serve to hold the hair temporarily play an important part. Corresponding substances for temporary reshaping usually contain synthetic polymers and/or waxes as the stabilising substance. Products for supporting the temporary reshaping of hair may be manufactured in the from of hairspray, hair wax, hair gel or hair mousse, for example.

The product for the temporary reshaping of hair, which are also called styling product in the following text, can be divided into various classes. These classes are categorised on the basis of the strength of hold, that is to say the property of lending hold to the hairstyle. A high hold strength usually makes it possible to stabilise the hairstyle for several hours up to a few days. In such cases, the hair is typically fixed so that it is prevented at least in part from adopting its natural movement behaviour. A low hold strength helps to shape a hairstyle, but lends it a predominantly natural appearance and a natural feel. The hairstyle is shaped with a certain flexibility and elasticity and retains its natural smoothness. Depending on the desired hairstyle, the hold strength can be adjusted precisely according to the desired hairstyle with the application of a corresponding styling product.

Besides the desired hold strength, styling product must also satisfy a whole range of other requirements. These can be defined roughly as properties on the hair, properties of the respective formulation, for example the properties of the foam, gel or sprayed aerosol, and properties relating to the handling of the styling product, wherein the properties on the hair are particularly important. These especially include moisture resistance, low tack and a balance conditioning effect. In addition, a styling product should also be usable for as many hair types as possible, and it should be gentle on the hair and skin. Besides this, however, the stability of the formulated styling product is also critically important.

Substances that contain water, particularly those substances which contain water in a form that is accessible for microorganisms, such as O/W emulsions, may generally be considered to be vulnerable to microbial colonisation. The removal of a part of the styling product directly from the packaging by hand can result in germs and bacteria being transferred to the styling product. To prevent microbial growth and bacterial propagation, preserving agents are added to the styling product.

Preservation with usual preserving agents is often not sufficient to protect the styling product from microbiological infection for the long term, particularly when water and large quantities (≥10 wt. %) of oils, particularly ester oils, are used. On the other hand, the use of preserving agents in high concentrations may affect the properties of the styling product adversely and thus also detract from its effect on the hair.

### BRIEF SUMMARY

This disclosure provides a cosmetic product for the temporary reshaping of keratinous fibres wherein the cosmetic product comprises
at least one ester oil which is liquid at about 25° C. and under a pressure of about 1013 mbar, wherein the total quantity of ester oil is from about 10 to about 70 wt. % relative to the total quantity of the cosmetic product,
at least one emulsifier,
one tropolone compound having formula (I)

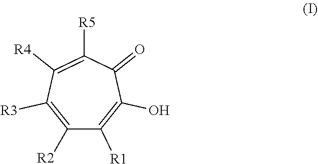

wherein the substituents R1, R2, R3, R4, R5 are independent of each other and each are: H, a linear or branched, saturated or unsaturated aliphatic hydrocarbon radical with up to about 30 C atoms, OH, OR6, wherein R6 is a linear or branched, saturated or unsaturated aliphatic hydrocarbon radical with up to about 30 C atoms, COOH, COOR7, wherein R7 is a linear or branched, saturated or unsaturated aliphatic hydrocarbon radical with up to about 30 C atoms, NO₂, NH₂, F, Cl, Br or I, and
water.

This disclosure also provides a method for treating and temporary reshaping of keratinous fibres, comprising the step of applying the cosmetic product to keratinous fibres.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

One of the objects of the present disclosure was to provide a cosmetic product for the temporary reshaping of keratinous fibres, containing water and high quantities (≥ about 10 wt. %) of ester oil, which is microbiologically non-hazardous with moderate preservation, enables good shaping of keratinous fibres, leaves the keratinous fibres feeling natural after application, and also enables natural appearance with a subtle lustre. It was a further object of the present disclosure to describe a use of the cosmetic product and a method for the treatment of keratinous fibres that may be applied simply and harmlessly.

The object is solved with a cosmetic product for the temporary reshaping of keratinous fibres containing:
at least one ester oil which is liquid at about 25° C. and under a pressure of about 1013 mbar, wherein the total quantity of ester oil is from about 10 to about 70 wt. %, preferably from about 12 to about 60 wt. %, more preferably from about 14 to about 50 wt. % and more preferably still from about 16 to about 40 wt. % relative to the total quantity of the cosmetic product,
at least one emulsifier,
one tropolone compound having formula (I)

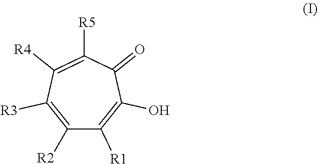

wherein the substituents R1, R2, R3, R4, R5 are independent of each other and have the following meaning: H, linear or branched, saturated or unsaturated aliphatic hydrocarbon radical with up to about 30 C atoms, OH, OR6, wherein R6 stands for a linear or branched, saturated or unsaturated aliphatic hydrocarbon radical with up to about 30 C atoms, COOH, COOR7, wherein R7 stands for a linear or branched, saturated or unsaturated aliphatic hydrocarbon radical with up to about 30 C atoms, NO₂, NH₂, F, Cl, Br or 1, and
water.

In this context, the term keratinous fibres as contemplated herein refers to hair, fur, wool and feathers, but particularly human hair.

For the purposes of the present disclosure, a liquid ester oil is understood to be a product that is flowable under normal conditions, that is to say at a temperature of about 25° C. and pressure of about 1013 mbar, as is clarified by its description as an “oil”. The ester oil is prepared from an acid and an alcohol and is not otherwise limited. Only phosphoric acid esters, sulfuric acid esters, alkoxylated carboxylic acids and sulfosuccinates are not to be included among the ester oils. The ester oil is thus exemplified by a lipophilic portion and, due to the carboxylate group, a certain hydrophilic portion as well. Due to its chemical structure, the ester oil is thus well suited for applying to keratinous fibres and spreading smoothly over the fibres without making the keratinous fibres heavy. This preserves the flexibility and elasticity of the keratinous fibres, which in turn allows the keratinous fibres to retain their texture, meaning that they do not feel artificial. The cosmetic product is practically undetectable on the keratinous fibres. The good distribution quality is supported by the flowability of the ester oil. Ester oils are also notable for their subtle glossiness, which they transfer to the keratinous fibres, lending them a natural appearance with good brightness and a “natural shine”.

In this context, it goes without saying that not only a single ester oil but two or more ester oils may also be used together.

In order to take full advantage of the positive properties of the ester oils, the at least one ester oil is used in a total quantity from about 10 to about 70 wt. %, preferably from about 12 to about 60 wt. %, more preferably from about 14 to about 50 wt. % and still more preferably from about 16 to about 40 wt. %, relative to the total quantity of the cosmetic product. In this context, with regard to the quantities of the ester oil it is understood that if two or more ester oils are used the aforementioned quantities are applicable for the total quantity of all ester oils.

In order to further improve the distribution quality of the cosmetic product, the cosmetic product also contains water, which serves as a solvent and is added in a desired quantity, which varies according to the application quantities of the cosmetic product. The total quantity of water relative to the total quantity of the cosmetic product is preferably from about 1 to about 89 wt. %, more preferably from about 20 to about 85 wt. %, still more preferably from about 30 to about 80 wt. % and particularly preferably from about 40 to about 70 wt. %.

In order to obtain a stable cosmetic product from the one or more ester oils and the water which does not tend towards syneresis, the cosmetic product also contains at least one emulsifier. The type and quantity of the emulsifier may vary according to the structure and total quantity of the ester oil on the one hand and the quantity of water on the other. By selecting a suitable emulsifier, the person skilled in the art can prepare the cosmetic product, e.g., as an emulsion, for example in the form of an oil-in-water (abbreviated to O/W) emulsion or a water-in-oil (abbreviated to W/O) emulsion, wherein O/W emulsions are preferred due to their application properties. In O/W emulsions, the aqueous phase is the outer phase. This is what makes it very easy to apply the O/W emulsion to keratinous fibres. After the application, the water evaporates and releases the inner phase, the oil phase, which then remains on the keratinous fibres and supports their long-term reshaping.

In principle, anionic, cationic, non-ionic and ampholytic surfactant compounds which in particular are suitable for use on the human body may be considered for use as emulsifiers. The ampholytic surfactant compounds contain zwitterionic surfactant compounds and ampholytes.

Non-ionic emulsifiers are preferred. In particular, adducts from the addition to ethylene oxide or propylene oxide of linear fatty alcohols, fatty acids, fatty acid alkanolamides, fatty acid monoglycerides, sorbitan fatty acid monoester, fatty acid glycerides, methylglucoside mono-fatty acid ester, or polydimethylsiloxanes and mixtures thereof are usable as non-ionic emulsifiers.

The at least one emulsifier is preferably selected from non-ionic emulsifiers such as adducts from about 2 to about 50 mol ethylene oxide with linear fatty alcohols with from about 8 to about 30, preferably from about 12 to about 18 carbon atoms, adducts from about 2 to about 50 mol ethylene oxide and from about 1 to about 5 mol propylene oxide with linear fatty alcohols having from about 8 to about 30 carbon atoms, preferably from about 12 to about 18 carbon atoms, adducts from about 2 to about 20 mol ethylene oxide with linear fatty acids having from about 12 to about 18 carbon atoms and mixtures thereof.

Examples of particularly preferred non-ionic tensides are compounds with the INCI designations Steareth-2, Steareth-21, Oleth-10, Ceteth-2, PEG-100 Stearate or PPG-5-Ceteth-20 and particularly combinations thereof. Particularly preferred is the combination of Oleth-10 and Ceteth-2, which forms the basis of particularly stable cosmetic products.

Other preferred emulsifiers are the esters of fatty acids having from about 12 to about 18 carbon atoms with saccharides. Particularly the mono- and/or diesters of sucrose with stearic and/or palmitic acid are favoured for use. Examples of particularly preferred non-ionic emulsifiers are compounds with the INCI designators Sucrose Stearate, Sucrose Distearate and mixtures thereof.

Further preferred emulsifiers are linear fatty acids having from about 12 to about 18 carbon atoms and mixtures thereof. The linear fatty acids may be present in the neutralised and/or non-neutralised state depending on the pH value.

Other preferred emulsifiers are adducts from about 2 to about 20 mol ethylene oxide with beeswax, e.g., in particular compounds with the INCI designators PEG-6 Beeswax, PEG-8 Beeswax, PEG-12 Beeswax or PEG-20 Beeswax. Of this class of emulsifiers, PEG-8 Beeswax is particularly preferred.

A further class of emulsifiers which are favoured for use in this application are the monoesters of fatty acids having from about 12 to about 22 carbon atoms with glycerol. The monoesters of glycerol with stearic and/or palmitic acid are particularly preferred for use. Examples of particularly preferred emulsifiers are compounds with the INCI designators Glyceryl Stearate, Glyceryl Palmitate or mixtures thereof.

The cosmetic product contains the at least one emulsifier preferably in a total quantity from about 1 to about 30 wt. %, more preferably from about 1.5 to about 20 wt. % and still more preferably from about 2 to about 15 wt. % relative to the total quantity of the cosmetic product.

Surprisingly, it has been found that the use of a tropolone compound lowers or inhibits the germ load of a cosmetic product containing water and at least about 10 wt. % ester oil, so that even after a prolonged storage time or period of use the bacterial load in the cosmetic product remains harmless and accordingly at a very low level. It has further been found that tropolone compounds in combination with the at least one liquid ester oil significantly increase the natural shine and natural texture of the keratinous fibres compared with cosmetic products that do not contain a tropolone compound. In particular, the natural elasticity and resilience of the keratinous fibres is preserved, with the result that the keratinous fibres retain their unaffected natural vitality and a highly attractive natural lustre after reshaping with the keratinous product.

Accordingly, the cosmetic product is exemplified by very good application and usage properties, it can be spread very well and evenly on keratinous fibres, it is non-hazardous from a microbiological perspective and it enables effective, flexible reshaping of the keratinous fibres without impairing the natural texture of the keratinous fibres. The keratinous fibres are given an agreeably soft, silky feel and the natural lustre of the keratinous fibres is particularly emphasised. At the same time, other properties that are typically required of cosmetic products for the temporary reshaping of keratinous fibres, such as low tack, are retained.

The tropolone compound used has the formula (I):

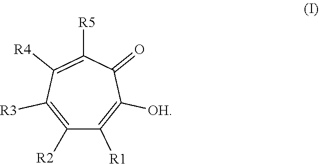

The substituents R1, R2, R3, R4, R5 are independent of each other and have the following meaning: H, linear or branched, saturated or unsaturated aliphatic hydrocarbon radical with up to about 30 C atoms, OH, OR6, wherein R6 stands for a linear or branched, saturated or unsaturated aliphatic hydrocarbon radical with up to about 30 C atoms, COOH, COOR7, wherein R7 stands for a linear or branched, saturated or unsaturated aliphatic hydrocarbon radical with up to about 30 C atoms, NO₂, NH₂, F, Cl, Br or I.

It is particularly preferred if the tropolone compound is tropolone (CAS no.: 533-75-5; 2-Hydroxy-2,4,6-cycloheptatrien-1-one).

According to a preferred embodiment, the total quantity of tropolone compound makes up from about 0.01 to about 2.0 wt. %, preferably from about 0.1 to about 1.5 wt. %, more preferably from about 0.2 to about 1.0 wt. % and still more preferably from about 0.25 to about 0.8 wt. % of the total quantity of the cosmetic product. Even with very low concentrations of tropolone, e.g., about 0.01 wt %, but particularly with concentrations higher than about 0.1 wt. %, the microbiological stability of the cosmetic product may be improved markedly and the natural lustre of the keratinous fibres may be increased while at the same time preserving good natural texture. On the other hand, high concentrations of more than about 2.0 wt. % do not lead to a further substantial improvement in terms of any of the aforementioned properties. For the purposes of good properties spectrum of the cosmetic product, a total quantity of from about 0.25 to about 0.8 wt. % tropolone is particularly preferred.

Because of very good application-related properties, including in particular a natural lustre and good silkiness without a tendency towards tack, the liquid ester oil is produced from at least one linear or branched, saturated or unsaturated mono-, di- or tricarboxylic acid having from 2 to about 30 carbon atoms, which may contain one or more hydroxyl groups, and at least one linear or branched, saturated or unsaturated alcohol having from 1 to about 30 carbon atoms. Alternatively or in addition thereto, the ester oil is selected from the group of: isopropyl myristate, isopropyl palmitate, isopropyl laurate, isopropyl lanolinate, isopropyl ricinoleate, isopropyl stearate, isononyl nonanoate, alkyl benzoate, dicaprylyl carbonate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-butyl stearate, n-hexyl laurate, n-decyl oleate, oleyl oleate, oleyl erucate, erucyl oleate, erucyl erucate and mixtures thereof.

Particularly advantageous properties of the cosmetic product are obtained when isopropyl myristate is used. The ester oil used then advantageously contains isopropyl myristate, wherein isopropyl myristate is particularly preferred as the sole liquid ester oil for the purposes of the present disclosure. Isopropyl myristate is an oil which spreads well on keratinous fibres, but at the same time is not so thin that it runs off the application site. In this way, the keratinous fibres are not weighed down and retain their natural texture. The flexibility of the keratinous fibres is preserved, while reshaping is good. Moreover, isopropyl myristate has a silky lustre which supports the natural shine of keratinous fibres particularly well. Furthermore, isopropyl myristate is a smooth oil without any tack which still adheres well to keratinous fibres, and thus makes long-term reshaping of keratinous fibres possible.

Also advantageously, the cosmetic product contains at least one natural or synthetic wax and/or at least one natural or synthetic fatty compound with a melting point higher than or equal to about 35° C. under pressure of about 1013 mbar, wherein the fatty compound is selected from saturated or unsaturated, linear or branched fatty acids having from 2 to about 30 carbon atoms which may optionally contain one or more hydroxyl groups, saturated or unsaturated, linear or branched fatty alcohols having from 2 to about 30 carbon atoms which may optionally contain one or more hydroxyl groups, and linear or branched fatty acid esters including at least one saturated or unsaturated, linear or branched mono-, di- or tricarboxylic acid having from 2 to about 30 carbon atoms, which may optionally contain one or more hydroxyl groups, and at least one saturated or unsaturated, linear or branched alcohol having from 1 to about 30 carbon atoms.

The addition of natural or synthetic wax and/or natural or synthetic fatty compound makes it possible not only to adjust the consistency of the cosmetic product but also to increase the hold strength of the keratinous fibres after the cosmetic product has been applied. In this way, the shape of the keratinous fibres is stabilised while retaining a high level of natural silkiness.

The total quantity of natural or synthetic waxes and/or natural or synthetic fatty compounds preferably makes up from about 2 to about 30 wt. %, preferably from about 5 to about 25 wt. %, more preferably from about 7 to about 20 wt. % and still more preferably from about 9 to about 15 wt. % of the total quantity of the cosmetic product in each case. With the preferred quantity ranges listed above, it is possible to support the natural texture of the keratinous fibres while retaining their natural lustre without substantially adding to the weight of the keratinous fibres or developing a tendency to tack.

Natural or synthetic waxes may be chosen from the group of solid paraffins or isoparaffins, vegetable waxes, candelilla wax, carnauba wax, esparto grass wax, Japan wax, cork wax, sugarcane wax, ouricury wax, montan wax, sunflower wax, fruit waxes and animal waxes, such as for example beeswaxes and other insect waxes, spermaceti, shellac wax, lanolin and uropygial fat, also mineral waxes such as ceresin and ozokerite or the petrochemical waxes such as petroleum jelly, paraffin waxes, microwaxes from polyethylene or polypropylene and polyethylene glycol waxes. It may be advantageous to use hydrated or hardened waxes. Chemically modified waxes, particularly the hard waxes such as montan ester waxes, sasol waxes and hydrated jojoba waxes are also usable.

Other suitable natural or synthetic waxes and/or natural or synthetic fatty compounds are triglyceride of saturated and optionally hydroxylated C16-30 fatty acids, for example hardened triglyceride fats (hydrated palm oil, hydrated coconut oil, hydrated castor oil), glyceryl tribehenate or glyceryl tri-12-hydroxystearate, also synthetic full esters from fatty acids and glycols (for example Syncrowax®) or polyols having 2-6 C atoms, fatty acid monoalkanolamides having a C12-22 acyl radical and a C2-4 alkanol radical, esters saturated and/or unsaturated, branched and/or unbranched alkane carboxylic acids with a chain length from about 1 to about 80 C atoms and saturated and/or unsaturated, branched and/or unbranched alcohols with a chain length from 1 to about 80 C atoms, including for example synthetic fatty acid-fatty alcohol esters such as stearyl stearate or cetyl palmitate, esters of aromatic carboxylic acids, dicarboxylic acids or hydroxycarboxylic acids (for example 12-hydroxystearic acid) and saturated and/or unsaturated, branched and/or unbranched alcohols having a chain length from about 1 to about 80 C atoms, lactides of long-chain hydroxycarboxylic acids and full esters from fatty alcohols and di- and tricarboxylic acids, such as dicetyl succinate or dicetyl-/stearyl adipate, and mixtures of these substances.

The natural or synthetic waxes and/or natural or synthetic fatty compounds may also be selected from the group of esters of saturated, unbranched alkane carboxylic acids having a chain length from about 14 to about 44 C atoms and saturated, unbranched alcohols having a chain length from about 14 to about 44 C atoms if the natural or synthetic wax and/or the natural or synthetic fatty compound or the total amount of the natural or synthetic waxes and/or natural or synthetic fatty compounds has a melting point equal to or higher than about 35° C. The natural or synthetic waxes and/or natural or synthetic fatty compounds may be chosen for example from the group of about C16-36 alkyl stearates, about C10-40 alkyl stearates, about C2-40 alkyl isostearates, about C20-40 dialkylesters of dimer acids, about C18-38 alkylhydroxy stearoyl stearates, about C20-40 alkyl erucates, and about C30-50 alkyl beeswax and cetearyl behenate are also usable. Silicone waxes such as stearyl trimethylsilane/stearyl alcohol may also be advantageous. Preferred wax components are the esters of saturated, monovalent about C20-C60 alcohols and saturated about C8-C30 monocarboxylic acids, particularly an about C20-C40 alkyl stearate.

Natural, chemically modified and synthetic waxes and/or natural, chemically modified and synthetic fatty compounds may be used alone or in combination. The teaching as contemplated herein thus also extends to the combined use of multiple waxes and/or fatty compounds. Additionally, a range of wax mixtures, possibly in a blend with further additives is also available commercially. The products designated “special wax 7686 OE” (a mixture of cetyl palmitate, beeswax, microcrystalline wax and polyethylene with a melting range from about 73 to about 75° C.; manufacturer: Kahl & Co), Polywax® GP 200 (a mixture of stearyl alcohol and polyethylene glycol stearate with a melting point from about 47 to about 51° C.; manufacturer: Croda) and “Weichceresin® FL 400” (a vaseline/vaseline oil/wax mixture with a melting point from about 50 to about 54° C.; manufacturer: Parafluid Mineralölgesellschaft) are examples of usable mixtures.

The wax or fatty compound is preferably selected from carnauba wax (INCI: Copernicia Cerifera Cera), fatty acids, and of these particularly palmitic acid and/or stearic acid, petroleum jelly and in particular mixtures of the aforementioned waxes and fatty compounds.

A further advantageous variant provides that the cosmetic product also contains at least one further preserving agent. A preserving agent that may be applied in cosmetics is used as the preserving agent. Phenoxyethanol has proven to be particularly suitable, as it seems to have a certain synergistic effect with the tropolone, with the result that only small quantities of the two raw materials need to be used to assure the microbiological harmlessness of the cosmetic product. The total quantity of additional preserving agent relative to the total quantity of the cosmetic product is advantageously from about 0.05 to about 2 wt. %, preferably from about 0.1 to about 0.9 wt. %, more preferably from about 0.2 to about 0.8 wt. %, and still more preferably from about 0.3 to about 0.6 wt. %. In these preferred quantity ranges, very effective preservation with regard to most germs in cosmetic products is achievable without producing any negative side effects.

Besides the aforementioned components which are used for preference in the cosmetic product, the cosmetic product may also include further usual cosmetic contents or components, which are employed particularly for use in styling products for reshaping keratinous fibres.

An example of a further advantageous component is a polyvalent alcohol. The total quantity of polyvalent alcohol particularly constitutes from about 0.5 to about 25 wt. % of the total quantity of the cosmetic product.

Preferred polyvalent alcohols are polyvalent C₂-C₉ alkanols having two to six hydroxyl groups and polyethylene glycols having from about 3 to about 20 ethylene oxide units. The product preferably contain at least one polyvalent C₂-C₉ alkanol having two to six hydroxyl groups or at least one water-soluble polyethylene glycol having from about 3 to about 20 ethylene oxide units or mixtures of at least one polyvalent C₂-C₉ alkanol having two to six hydroxyl groups and at least one water-soluble polyethylene glycol having from about 3 to about 20 ethylene oxide units.

The C₂-C₉ alkanols having two to six hydroxyl groups are preferably selected from 1,2-propylene glycol, 1,3-propylene glycol, 2-methyl-1,3-propanediol, glycerol, 1,2-butylene glycol, 1,3-butylene glycol, 1,4-butylene glycol, 1,2-pentanediol, 1,5-pentanediol, 1,2-hexanediol, 1,6-hexanediol, 1,2,6-hexanetriol, 1,2-octanediol, 1,8-octanediol, dipropylene glycol, tripropylene glycol, diglycerol, triglycerol, erythritol, sorbitol, cis-1,4-dimethylol cyclohexane, trans-1,4-dimethylol cyclohexane, any isomeric mixtures of cis- and trans-1,4-dimethylol cyclohexane and mixtures of said polyvalent alcohols. Suitable polyethylene glycols are selected from PEG-4, PEG-6, PEG-7, PEG-8, PEG-9, PEG-10, PEG-12, PEG-14, PEG-16, PEG-18 PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, PEG-350, PEG-400, PEG-500, PEG-800, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-180M and mixtures thereof.

Further suitable polyvalent alcohols are methylol compounds, such as in particular trimethylolethane, trimethylolpropane, trimethylolbutane, pentaerythritol and dipentaerythritol, sugar alcohols with from about 5 to about 12 carbon atoms, such as sorbitol or mannitol, sugars having from about 5 to about 12 carbon atoms such as glucose or sucrose, or amino sugars such as glucamine. Another suitable polyvalent alcohol is dexpanthenol (INCI: Panthenol).

Most particularly preferred polyvalent alcohols are selected from the group of 1,2-propylene glycol, 1,3-propylene glycol, 1,2-butylene glycol, 1,3-butylene glycol, 1,4-butylene glycol, glycerol, panthenol (INCI), PEG-45M and mixtures thereof.

Preferred cosmetic products contain the one or more polyvalent alcohols in a total quantity from about 1 to about 15 wt. % and more preferably in a total quantity from about 2 to about 10 wt. % relative to the total quantity of the cosmetic product.,

The cosmetic product may further contain at least one film-forming polymer. Examples of film-forming polymers are cationic, anionic, non-ionic and amphoteric polymers. The cosmetic product may contain the at least one film-forming polymer preferably in a total quantity from about 1 to about 60 wt. %, preferably from about 1.5 to about 20 wt. % and more preferably from about 2 to about 15 wt. % relative to the total quantity of the cosmetic product.

Examples of particularly suitable film-forming polymers include, according to their INCI designators: Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVPNA/Itaconic Acid Copolymer, PVPNA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VPNA Copolymer, VPNinyl Caprolactam/DMAPA Acrylates Copolymer, Yeast Palmitate, Styrene/VP Copolymer, Cellulose ethers such as hydroxypropyl cellulose, hydroxyethyl cellulose and methyl hydroxypropyl cellulose, such as are offered for sale commercially under the brand names Culminal and Benecel (AQUALON).

Siloxanes are also suitable for use as film-forming polymers. Such siloxanes may be either water-soluble or water-insoluble. Both volatile and non-volatile siloxanes are suitable, wherein the term non-volatile siloxanes is understood to refer to those compounds whose boiling point is higher than about 200° C. under normal pressure of about 1013 mbar. Preferred siloxanes are polydialkyl siloxanes such as polydimethyl siloxane, polyalkylaryl siloxanes such as polyphenylmethyl siloxane, ethoxylated poly-dialkyl siloxanes and polydialkyl siloxanes that contain amino and/or hydroxy groups. Glycosidically substituted silicones are may also be considered.

Homopolyacrylic acid (INCI: Carbomer), available commercially in several different variations with the trade name Carbopol®, is suitable for use as a film-forming polymer.

The film-forming polymer is preferably selected from vinylpyrrolidone-containing polymers. The film-forming polymer is particularly preferably selected from the group of polyvinyl pyrrolidone, vinylpyrrolidone-vinylacetate-copolymer, vinyl caprolactam/NP/dimethylaminoethyl methacrylate copolymer (INCI), VP/DMAPA Acrylates Copolymer (INCI) and mixtures thereof.

Another preferred film-forming polymer is Octylacrylamide/Acrylates/Butylaminoethyl Methacrylate Copolymer (INCI), which is marketed commercially by AkzoNobel with trade name “Amphomer®”.

Care components such as hydrocarbon oils are also noteworthy in particular as further suitable adjuvants and additives.

Preferred hydrocarbon oils are selected from natural and synthetic hydrocarbons, particularly preferably from mineral oils, paraffin oils, C₁₈-C₃₀ isoparaffins, in particular isoeicosane, polyisobutene and dolydecene, which are available commercially under the trade names Emery® 3004, 3006, 3010 or Ethylflo® from Albemarle or Nexbase® 2004G from Nestle, for example, further selected from C₈-C₁₆ isoparaffins, particularly from isodecane, isododecane, isotetradecane and isohexadecane, and mixtures thereof as well as 1,3-di-(2-ethylhexyl)-cyclohexane (available commercially for example from BASF under the trade name Cetiol® S).

The product may also contain for example at least one protein hydrolysate and/or one of the derivatives thereof as a care component. Protein hydrolysates are product mixtures which are obtained by the acid-, alkali- or enzyme-catalysed decomposition of proteins. The term protein hydrolysates is also understood to include total hydrolysates and individual amino acids and derivatives thereof as well as mixtures of different amino acids. The molecular mass of the protein hydrolysates that may be used is between from about 75, the molecular mass of glycine, and about 200,000, the molecular mass is preferably from about 75 to about 50,000 and most particularly preferably about 75 to about 20,000 Dalton.

The cosmetic product may further contain at least one vitamin, one provitamin, one vitamin precursor and/or one of the derivatives thereof as a care component. In this context, those vitamins, provitamins and vitamin precursors which are typically classified in groups A, B, C, E, F and H are preferred.

The cosmetic product may also contain neutralisers or pH-adjusting agents in order to adjust its pH value. Examples of neutralisers used in cosmetic products are primary aminoalcohols such as Aminomethyl Propanol (INCI), which is available commercially under the trade name AMP-ULTRA® PC, for example AMP-ULTRA® PC 2000.

The cosmetic product may also contain at least one chromophoric substance, for example one or more colouring agents and/or one or more pigments for aesthetic purposes. There are no individual restrictions regarding the colouring agents, and these may be selected freely from the colouring substances that permitted for use in cosmetics. The total quantity of colouring agents is variable according to the degree of colouring desired for the cosmetic product.

The present disclosure also relates to the cosmetic, non-therapeutic use of the cosmetic product disclosed in the preceding text for the temporary reshaping, also called “styling”, of keratinous fibres, and alternatively or additionally for improving the natural lustre and/or the natural texture of keratinous fibres, the keratinous fibres being in particular human hair.

A further object of the present disclosure is the use of a tropolone compound having formula (I)

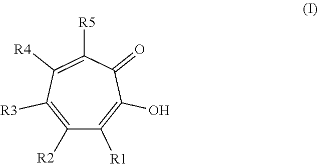

wherein the substituents R1, R2, R3, R4, R5 are independent of each other and have the following meaning: H, linear or branched, saturated or unsaturated aliphatic hydrocarbon radical with up to about 30 C atoms, OH, OR6, wherein R6 stands for a linear or branched, saturated or unsaturated aliphatic hydrocarbon radical with up to about 30 C atoms, COOH, COOR7, wherein R7 stands for a linear or branched, saturated or unsaturated aliphatic hydrocarbon radical with up to about 30 C atoms, NO₂, NH₂, F, Cl, Br or I for preserving a cosmetic product for the temporary reshaping of keratinous fibres, containing:
at least about 10 wt. % of an ester oil which is liquid at about 25° C. and under pressure of about 1013 mbar, and water.

Also disclosed is a method for treating and particularly for the temporary reshaping of keratinous fibres, particularly human hair, in which the cosmetic product described above is applied to keratinous fibres. The method is simple to apply and enables effective reshaping of keratinous fibres.

### Example

The present disclosure is illustrated by the following example, which is not to be considered limiting thereof in any way.

The following cosmetic product was manufactured:

| | | Example 1 |
|---|---|---|
| Component/Raw material | INCI designation | (wt. %) |
| Carnauba wax | Copernicia | 3 |
| | Cerifera Cera | |
| Weichceresin FL 400 | Petroleum jelly | 4 |
| Edenor L2SM | Palmitic Acid, | 5 |
| | Stearic Acid | |
| Isopropyl myristate | Isopropyl | 25 |
| | Myristate | |
| Oleyl alcohol 10 EO | Oleth-10 | 3 |
| Ceteth-2 | Ceteth-2 | 2 |
| 2-Phenoxyethanol | Phenoxyethanol | 0.5 |
| Tropolone | Tropolone | 0.5 |
| Water | Water | to 100 |

The quantities shown in the table above are expressed in wt. % of the respective raw material relative to the total quantity of the cosmetic product.

The cosmetic product 1 was physically and microbiologically stable over a period of 12 weeks at various temperatures (room temperature, 0° C., 45° C., −10° C.). The cosmetic product 1 manifested good applicability and particularly good spreadability by hand. The hair could be reshaped very well with the cosmetic product 1 without becoming heavier. Its natural texture and natural lustre were enhanced by the cosmetic product 1.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product for the temporary reshaping of keratinous fibres comprising:
at least one ester oil which is liquid at about 25° C. and under a pressure of about 1013 mbar, wherein the total quantity of ester oil is from about 10 to about 70 wt. % relative to the total quantity of the cosmetic product,
at least one emulsifier,
one tropolone compound having formula (I) 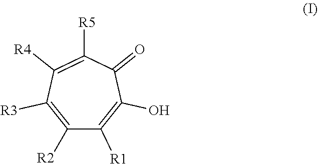
wherein the substituents R1, R2, R3, R4, R5 are independent of each other and each are: H, a linear or branched, saturated or unsaturated aliphatic hydrocarbon radical with up to about 30 C atoms, OH, OR6, wherein R6 is a linear or branched, saturated or unsaturated aliphatic hydrocarbon radical with up to about 30 C atoms, COOH, COOR7, wherein R7 is a linear or branched, saturated or unsaturated aliphatic hydrocarbon radical with up to about 30 C atoms, NO₂, NH₂, F, Cl, Br or I, and
water.

2. The cosmetic product according to claim 1, wherein the total quantity of tropolone compound makes up from about 0.01 to about 2.0 wt. % of the total quantity of the cosmetic product.

3. The cosmetic product according to claim 1, wherein the the liquid ester oil is produced from at least one linear or branched, saturated or unsaturated mono-, di- or tricarboxylic acid having from 2 to about 30 carbon atoms, and at least one linear or branched, saturated or unsaturated alcohol having from 1 to about 30 carbon atoms.

4. The cosmetic product according to claim 1, wherein the ester oil comprises isopropyl myristate.

5. The cosmetic product according to claim 1, further comprising at least one natural or synthetic wax and/or at least one natural or synthetic fatty compound with a melting point higher than or equal to about 35° C. under pressure of about 1013 mbar, wherein the fatty compound is selected from
saturated or unsaturated, linear or branched fatty acids having from 2 to about 30 carbon atoms,
saturated or unsaturated, linear or branched fatty alcohols having from 2 to about 30 carbon atoms, and linear or branched fatty acid esters comprising at least one saturated or unsaturated or unsaturated group,
linear or branched mono-, di- or tricarboxylic acids having from 2 to about 30 carbon atoms, and at least one saturated or unsaturated, linear or branched alcohol having from 1 to about 30 carbon atoms.

6. The cosmetic product according to claim 5, wherein the total quantity of wax and/or fatty compound makes up from about 2 to about 30 wt. % of the total quantity of the cosmetic product.

7. The cosmetic product according to claim 1, further comprising at least one further preserving agent, wherein the total quantity of the additional preserving agent relative to the total quantity of the cosmetic product is from about 0.05 to about 2 wt. %.

8. (canceled)

9. (canceled)

10. A method for treating and temporary reshaping of keratinous fibres, comprising applying the cosmetic product according to claim 1 to keratinous fibres.

11. The cosmetic product according to claim 1, wherein the total quantity of the ester oil is from about 12 to about 60 wt. % relative to the total quantity of the cosmetic product.

12. The cosmetic product according to claim 1, wherein the total quantity of the ester oil is from about 14 to about 50 wt. % relative to the total quantity of the cosmetic product.

13. The cosmetic product according to claim 1, wherein the total quantity of the tropolone compound makes up from about 0.1 to about 1.5 wt. % of the total quantity of the cosmetic product.

14. The cosmetic product according to claim 1, wherein the total quantity of the tropolone compound makes up from about 0.2 to about 1.0 wt. % of the total quantity of the cosmetic product.

15. The cosmetic product according to claim 1, wherein the total quantity of the tropolone compound makes up from about 0.25 to about 0.8 wt. % of the total quantity of the cosmetic product.

16. The cosmetic product according to claim 1, wherein the the liquid ester oil is selected from the group of: isopropyl myristate, isopropyl palmitate, isopropyl laurate, isopropyl lanolinate, isopropyl ricinoleate, isopropyl stearate, isononyl nonanoate, alkyl benzoate, dicaprylyl carbonate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-butyl stearate, n-hexyl laurate, n-decyl oleate, oleyl oleate, oleyl erucate, erucyl oleate, erucyl erucate and mixtures thereof.

17. The cosmetic product according to claim 1, wherein the ester oil is isopropyl myristate.

18. The cosmetic product according to claim 1, further comprising at least one natural or synthetic wax and/or at least one natural or synthetic fatty compound with a melting point higher than or equal to about 35° C. under pressure of about 1013 mbar, wherein the fatty compound is selected from
saturated or unsaturated, linear or branched fatty acids having from 2 to about 30 carbon atoms and comprising one or more hydroxyl groups,
saturated or unsaturated, linear or branched fatty alcohols having from 2 to about 30 carbon atoms comprising one or more hydroxyl groups, and linear or branched fatty acid esters comprising at least one saturated or unsaturated group,
linear or branched mono-, di- or tricarboxylic acid having from 2 to about 30 carbon atoms comprising one or more hydroxyl groups, and at least one saturated or unsaturated, linear or branched alcohol having from 1 to about 30 carbon atoms.

19. The cosmetic product according to claim 5, wherein the total quantity of wax and/or fatty compound makes up from about 5 to about 25 wt. % of the total quantity of the cosmetic product.

20. The cosmetic product according to claim 5, wherein the total quantity of wax and/or fatty compound makes up from about 7 to about 20 wt. % of the total quantity of the cosmetic product.

21. The cosmetic product according to claim 5, wherein the total quantity of wax and/or fatty compound makes up from about 9 to about 15 wt. % of the total quantity of the cosmetic product.

22. The cosmetic product according to claim 1, further comprising phenoxyethanol, wherein the total quantity of the phenoxyethanol relative to the total quantity of the cosmetic product is from about 0.3 to about 0.6 wt. %.
